(19) United States
(12) Patent Application Publication
(10) Pub. No.: US 2004/0098023 A1
(43) Pub. Date: May 20, 2004

(54) Embolic device made of nanofibers

(75) Inventors: Elaine Lee, Sunnyvale, CA (US); Paul Steven Seifert, Oregon House, CA (US)

Correspondence Address: Bingham McCutchen, LLP, Suite 1800, Three Embarcadero, San Francisco, CA, 94111-4067, US

(73) Assignee: Scimed Life Systems, Inc.

(21) Appl. No.: 10/295,727

(22) Filed: Nov. 15, 2002

(51) Int. Cl. A61M 29/00

(52) U.S. Cl. 606/200

(57) ABSTRACT

Vaso-occlusive devices for occlusion of a body cavity are provided. The vaso-occlusive devices include a core member and a fibrous structure coupled to the core member. The fibrous structure comprises strands of nanofibers. Methods of using the vaso-occlusive devices are also described.

### BACKGROUND OF THE INVENTION

1. Field of the Invention

The field of the invention pertains to implantable devices, and, more particularly, vaso-occlusive devices for the occlusion of body lumens and cavities.

2. Background of the Invention

In many clinical situations, blood vessels are occluded for a variety of purposes, such as to control bleeding, to prevent blood supply to tumors, and to block blood flow within an aneurysm, arteriovenous malformation, or arteriovenous fistula.

Embolization of blood vessels is particularly useful in treating aneurysms. Aneurysms are abnormal blood filled dilations of a blood vessel wall, which may rupture causing significant bleeding. For the cases of intracranial aneurysms, the significant bleeding may lead to damage to surrounding brain tissue or death. Intracranial aneurysms may be difficult to treat when they are formed in remote cerebral blood vessels, which are very difficult to access. If left untreated, hemodynamic forces of normal pulsatile blood flow can rupture fragile tissue in the area of the aneurysm causing a stroke.

Vaso-occlusive devices have been used in the treatment of aneurysms. Vaso-occlusive devices are surgical implants placed within blood vessels or vascular cavities, typically by using a catheter to form a thrombus and occlude the site. For instance, a stroke or other such vascular accident may be treated by placing a vaso-occlusive device proximal of the site to block the flow of blood to the site and alleviate the leakage. An aneurysm may similarly be treated by introducing a vaso-occlusive device through the neck of the aneurysm. The thrombogenic properties of the vaso-occlusive device cause a mass to form in the aneurysm and alleviate the potential for growth of the aneurysm and its subsequent rupture. Other diseases, such as tumors, may often be treated by occluding the blood flow to the tumor.

There are a variety of vaso-occlusive devices suitable for forming thrombi. One such device is found in U.S. Pat. No. 4,994,069, to Ritchart et al., the entirety of which is incorporated by reference. That patent describes a vaso-occlusive coil that assumes a linear helical configuration when stretched and a folded convoluted configuration when relaxed. The coil has a stretched configuration when placed in a catheter, which is used in placement of the coil at the desired site, and assumes the convoluted configuration when the coil is ejected from the catheter and the coil relaxes. Ritchart et al. describes a variety of shapes, including “flower” shapes and double vortices. A random shape is described as well.

U.S. Pat. No. 6,280,457B1 to Wallace et al., describes an occlusive device including an inner core wire covered with a polymer. The polymeric material includes protein based polymers, absorbable polymers, non-protein based polymers, and combinations thereof. The polymer facilitates forming of emboli to occlude a body cavity.

Vaso-occlusive coils having complex, three-dimensional structures in a relaxed configuration are described in U.S. Pat. No. 6,322,576B1 to Wallace et al. The coils may be deployed in the approximate shape of a sphere, an ovoid, a clover, a box-like structure or other distorted spherical shape. The patent also describes methods of winding the anatomically shaped vaso-occlusive device into appropriately shaped forms and annealing them to form various devices.

Vaso-occlusive coils having little or no inherent secondary shape have also been described. For instance, U.S. Pat. Nos. 5,690,666 and 5,826,587 both by Berenstein et al. describe coils having little or no shape after introduction into the vascular space.

There are a variety of ways of discharging shaped coils and linear coils into a body cavity. In addition to those patents that describe physically pushing a coil out of the catheter into the body cavity (e.g., Ritchart et al.), there are a number of other ways to release the coil at a specifically chosen time and site. U.S. Pat. No. 5,354,295 and its parent, U.S. Pat. No. 5,122,136, both to Guglielmi et al., describe an electrolytically detachable embolic device.

A variety of mechanically detachable devices are also known. Various examples of these devices are described in U.S. Pat. No. 5,234,437, to Sepetka, U.S. Pat. No. 5,250,071 to Palermo, U.S. Pat. No. 5,261,916, to Engelson, U.S. Pat. No. 5,304,195, to Twyford et al., U.S. Pat. No. 5,312,415, to Palermo, and U.S. Pat. No. 5,350,397, to Palermo et al.

When the above-mentioned vaso-occlusive devices are placed within an aneurysm, they tend to induce the formation of fibrin network (clot or thrombus), which serves as a temporary scaffold. This scaffold provides a high-surface-area substrate on which the cells responsible for wound healing (such as fibroblasts) migrate and proliferate as they deposit collagen to replace the clot with more stable collagenous fibrous tissue. However, the enzymes present in the blood could break down the fibrin clot too quickly in relation to the rate of collagen deposition, thus limiting the movement and growth of the wound-healing cells. As a result, the thrombus formed within the aneurysm may develop voids and/or may not have the sufficient size to completely occlude the aneurysm.

Accordingly, devices and methods for occluding aneurysms or other body cavities would be useful.

### SUMMARY OF THE INVENTION

The present invention is directed to vaso-occlusive devices that may be deployed within the vasculature of a patient to occlude the flow of blood therein, and to methods for using such devices. Preferably, the vaso-occlusive device may be deployed to generate emboli in aneurysms located within the vasculatures of humans, but may also be used at any site in a human or animal that requires occlusion.

In accordance with one aspect of the present invention, a vaso-occlusive device may include a core member and a fibrous structure coupled to the core member. The fibrous structure, which may be fabricated, for example, by an electrospinning process, may include strands of non-woven fibers having nanometer-scale diameters. The architecture of the fibrous structure may provide a high level of surface area to which cells may attach, and may provide a stable scaffold for filling an aneurysm. The core member may provide a grid onto which the fibrous structure may be disposed. Depending on the material from which the core member is made, the core member may also enhance the rigidity of the vaso-occlusive device. The vaso-occlusive device may be carried to the target site using a catheter and released therefrom using any one of a variety of detachable means, such as an electrolytic joint or a mechanical joint.

The vaso-occlusive device may have a relaxed configuration that may assume a variety of shapes. For example, the vaso-occlusive device may have a substantially linear or curvilinear (slightly curved, i.e. having less than 360° spiral) relaxed configuration. Alternatively, the vaso-occlusive device may assume a secondary relaxed shape formed by wrapping a core member having a primary shape that is substantially linear around a shaping element. The secondary shape may be a helical coil or other shapes. As a further alternative, the vaso-occlusive device may also assume a tertiary relaxed shape formed by wrapping a core member having a secondary shape around a shaping element. The tertiary shape may be, for example, in a shape of a clover leaf, a twisted figure-8, a flower, a sphere, a vortex, an ovoid, or random shapes.

Other aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a side view of a first preferred embodiment of a vaso-occlusive device in accordance with the present invention, including a fibrous structure disposed around a core member;

FIG. 1A is a detail of one end of the device of FIG. 1;

FIG. 2 diagrams an electrospinning apparatus;

FIGS. 3-8 are side views of variations of a vaso-occlusive device in accordance with the present invention;

FIGS. 9 and 10 show examples of a vaso-occlusive device having a secondary shape;

FIGS. 11-17 show examples of a vaso-occlusive device having a tertiary shape;

FIG. 18 is a side view of a vaso-occlusive device being delivered within a body cavity using a delivery catheter;

FIG. 19 is a cross-sectional side view of a vaso-occlusive device being delivered using a delivery catheter, showing the vaso-occlusive device having a stretched configuration when resided within the delivery catheter, and assuming a secondary shape when unrestrained outside the delivery catheter;

FIG. 20 is a cross-sectional side view of a vaso-occlusive device being delivered using a delivery catheter, showing the vaso-occlusive device maintaining a secondary shape inside the delivery catheter;

FIG. 21 is a cross-sectional side view of a vaso-occlusive device being delivered using a delivery catheter, showing the vaso-occlusive device changing from a secondary shape to a tertiary shape as it exits from the delivery catheter;

FIG. 22 is a side view of a portion of a delivery catheter from which a vaso-occlusive device is deployed and mechanically released; and

FIG. 23 is a side view of a portion of a delivery catheter from which a vaso-occlusive device is deployed and electrolytically released.

### DETAILED DESCRIPTION OF THE PREFERRED EMBODIMENTS

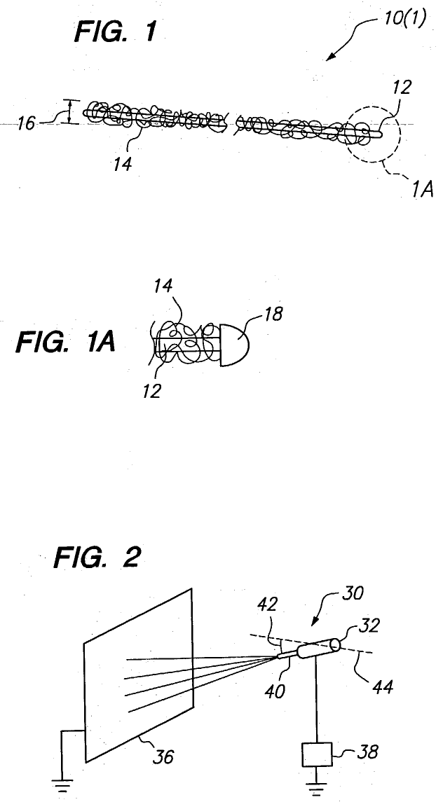

Turning to the drawings, FIGS. 1 and 3-8 show various embodiments of a vaso-occlusive device 10, in accordance with the present invention. Generally, the vaso-occlusive device 10 includes a core member 12 and a fibrous structure 14 carried by the core member 12. The core member 12 may provide a grid to which the fibrous structure 14 may be attached. Depending upon the material from which the core member 12 is made, the core member 12 may also provide a desired rigidity for the vaso-occlusive device 10. The fibrous structure 14, which includes one or more nano-scale fibers (nanofibers), may provide or enhance thrombogenic properties of the vaso-occlusive device 10. The term, “nano-scale fiber” or “nanofibers,” refers to fiber that has a diameter or cross-sectional dimension in the range from about 50 to 10000 nm. The fibrous structure 14 would be discussed in further detail below. As shown in FIG. 1, the vaso-occlusive device 10 has an overall diameter or cross-section 16, which is preferably in the range of 0.01 inch to 0.015 inch. However, the vaso-occlusive device 10 may have other diameters as well. The vaso-occlusive device 10 may optionally include an end cap 18, as shown in FIG. 1A.

The core member 12 preferably has a circular cross-sectional shape. Alternatively, the core member 12 may have a rectangular, triangular, or other geometric cross-section. In a further alternative, the core member 12 may have an irregular shaped cross-section. The core member 12 is preferably made of a biodegradable material. Biodegradable or absorbable materials suitable for the core member 12 may include, but are not limited to, synthetic polymers, polysaccharides, and proteins. Suitable polymers may include, for example, polyglycolic acid, polylactic acid, polycaprolactone, polyhydroxybutyrate, polyhydroxyvalerate, polydioxanone, polycarbonates, polyanhydrides, polyhydroxyalkanoates, polyarylates, polysaccharides, polyamino acids, and copolymers thereof.

In addition or alternatively, proteins may be used, such as collagen, elastin, fibrin, fibrinogen, fibronectin, vitronectin, laminin, silk, and/or gelatin. In addition or alternatively, polysaccharides may be used, such as chitin, chitosan, cellulose, alginate, hyaluronic acid, and chondroitin sulfate. Many of these materials are commercially available. Fibrin-containing compositions are commercially available, for example from Baxter. Collagen-containing compositions are commercially available, for example, from Cohesion Technologies, Inc., of Palo Alto, Calif. Fibrinogen-containing compositions are described, for example, in U.S. Pat. Nos. 6,168,788 and 5,290,552, the disclosure of which is expressly incorporated herein by reference. As will be readily apparent, absorbable materials may be used alone or in any combination with each other. The absorbable material may be a mono-filament or multifilament strands.

Furthermore, the absorbable materials may be used in combination with additional components. For example, lubricious materials (e.g., hydrophilic) materials may be used to coat the core member 12. One or more bioactive materials may also be included in the composition of the core member 12. The term “bioactive” includes any agent that exhibits effects in vivo, for example a thrombotic agent, a therapeutic agent, and the like. Examples of bioactive materials include cytokines; extracellular matrix molecules (e.g., collagen or fibrin); matrix metalloproteinase inhibitors; trace metals (e.g., copper); other molecules that may stabilize thrombus formation or inhibit clot lysis (e.g., proteins, including Factor XIII, α₂-antiplasmin, plasminogen activator inhibitor-1 (PAI-1), and the like); and their functional fragments (e.g., the P1 or P2 epitopes of fibrin). Examples of cytokines that may be used alone or in combination with other compounds may include basic fibroblast growth factor (bFGF), platelet derived growth factor (PDGF), vascular endothelial growth factor (VEGF), transforming growth factor beta (TGF-β), and the like. Cytokines, extracellular matrix molecules, matrix metalloproteinase inhibitors, and thrombus stabilizing molecules are commercially available from several vendors, such as Genzyme (Framingham, Mass.), Genentech (South San Francisco, Calif.), Amgen (Thousand Oaks, Calif.), R&D Systems, and Immunex (Seattle, Wash.). Additionally, bioactive polypeptides that may be synthesized recombinantly as the sequence of many of these molecules are also available, for example, from the GenBank database. Thus, it is intended that the core member 12 may include use of DNA or RNA encoded bioactive molecules. Furthermore, molecules having similar biological activity as wild-type or purified cytokines, extracellular matrix molecules, matrix metalloproteinase inhibitors, thrombus-stabilizing proteins (e.g., recombinantly produced or mutants thereof), and nucleic acid encoding these molecules may also be used. The amount and concentration of the bioactive materials that may be included in the composition of the core member 12 may vary depending upon the specific application, and may be readily determined by one skilled in the art. It will be understood that any combination of materials, concentration, and/or dosage may be used, so long as it is not harmful to the subject.

The core member 12 may also include one or more radiopaque materials for visualizing the vaso-occlusive members 12 in situ. For example, the core member 12 may be coated or mixed with radiopaque materials such as metals (e.g. tantalum, gold, tungsten or platinum), barium sulfate, bismuth oxide, bismuth subcarbonate, and the like. Alternatively, continuous or discrete radiopaque markers may be affixed to the core member 12.

Alternatively, the core member 12 may be made of non-biodegradable materials, such as metals, which may be more elastic than the biodegradable materials described previously. Suitable metals and alloys for the core member 12 may include the Platinum Group metals, especially platinum, rhodium, palladium, rhenium, as well as tungsten, gold, silver, tantalum, and alloys of these metals. These metals have significant radiopacity and their alloys may be tailored to accomplish an appropriate blend of flexibility and stiffness. They are also largely biologically inert. Additional coating materials, such as a polymer, and/or biodegradable material, such as discussed previously, may be added to the surface of the core member 12 to improve the thrombogenic or other properties of the vaso-occlusive device. The core member 12 may also be formed from stainless steels if some sacrifice of radiopacity may be tolerated.

Other materials that may be used may include “super-elastic alloys,” such as nickel/titanium (“Nitinol”) alloys, copper/zinc alloys, or nickel/aluminum alloys. Exemplary alloys that may be used are described in U.S. Pat. Nos. 3,174,851, 3,351,463, and 3,753,700, the disclosures of which are expressly incorporated herein by reference. If Nitinol is used, the diameter of the core member 12 may be significantly smaller than that of a core member 12 made from relatively more ductile platinum or platinum/tungsten alloy.

The core member 12 may also be made of radiolucent fibers or polymers (or metallic threads coated with radiolucent or radiopaque fibers), such as Dacron (polyester), polyglycolic acid, polylactic acid, fluoropolymers (polytetrafluoroethylene), Nylon (polyamide), and/or silk.

The fibrous structure 14 generally includes one or more strands of fibers having nanometer-scale diameters (“nanofibers”). The strands of fibers are preferably non-woven. The fibrous structure 14 may be fabricated at least in part by an electrospinning process or technique, such as that described in U.S. Pat. No. 1,975,504, the disclosure of which is expressly incorporated herein by reference. FIG. 2 shows an example of en electrospinning apparatus 30, which includes a syringe 32 containing a polymer solution 34 (not shown), a copper collecting plate 36, and a power supply 38. The syringe 32 is preferably a 20-mL glass syringe fitted with a needle 40. The needle 40 is preferably an eighteen gage (18GA) needle, but may also be any tubular element capable of carrying out the function(s) described herein. The polymer solution 34 is preferably prepared by dissolving one gram (1 g) of copolymer poly (D, L-lactide-coglycolide) (PLGA) (Purac, Lincolnshire, Ill.) in twenty milliliters (20 mL) of organic solvent mixture composed of (1:1) tetrahydrofuran (THF; Fisher, Pittsburgh, Pa.) and dimethylformamide (DMF; Sigma, St. Louis, Mo.) and mixing it well by vortexing the mixture overnight.

The polymer solution 34 may also be prepared using other polymers, such as polyethylene oxide (PEO), acrylic, nylon, polyethylene glycol (PEG), polyacrylonitrile (PAN), polyethylene terephthalate (PET), poly (p-phenylene terephthalamide) (PPTA), and the like. Degradable polymers may also be used, which include polyglycolic acid, polylactic acid, polycaprolactone, polyhydroxybutyrate, polyhydroxyvalerate, polydioxanone, polycarbonates, polyanhydrides, polyhydroxyalkanoates, polyarylates, polysaccharides, polyamino acids, and copolymers thereof. Other polymer solutions 34 known in the art may be also be used, including proteins such as collagen, elastin, fibrin, fibrinogen, fibronectin, vitronectin, laminin, silk, and/or gelatin. Furthermore, any of the bioactive materials discussed previously with reference to the core member 12 may also be included in the polymer solutions 34. Alternatively, the bioactive materials may also be added to the fibrous structure 14 after the fibrous structure 14 is formed. The bioactive materials may be attached to the fibrous structure 14 chemically, or the fibrous structure 14 may be fully or partially filled (or soaked) with a solution containing the bioactive materials.

During the process of electrospinning, the syringe 32 is directed at an angle 42, such as a 45-degree angle, down-tilted from the horizontal 44, towards the copper collecting plate 36. The tip of the needle 40 is preferably placed twenty centimeters (20 cm) from the copper collecting plate 36. It should be understood by those skilled in the art that the syringe 32 may be oriented at different angles 42 from the horizontal 44, and positioned at different distance from the copper collecting plate 36, depending on the particular application. When the power supply 38 supplies a voltage (preferably eighteen kilovolts), the copper collecting plate (cathode) becomes negatively charged, and the needle 40 (anode) of the syringe 32 becomes positively charged. The combining force of gravity and the created electrostatic charge then causes the polymer solution 34 to be drawn from the syringe 32, forming a pendant drop at the tip of the needle 40. A positive-charged jet is then ejected from the drop and is splayed to a negative-charged target on the copper collecting plate 36. As a result, the fibrous structure 14 is formed on the copper collecting plate 36, and is then carefully removed for subsequent use. It should be noted that the polarity of the charges on the needle 40 and the plate 36 may be switched. Other techniques known in the art for fabricating fibrous elements may also be used to produce the fibrous structure 14.

The fibrous structure 14 produced by the electrospinning process is generally composed of non-woven and randomly oriented fibers having diameters or cross-sections in the range from 100 to 5000 nm. Such architecture of the fibrous structure 14, which has been found to promote cell growth, is similar to those of some natural extracellular matrices (ECM). ECM, which surround cells to provide mechanical support, are primarily composed of fibrous proteins of nanometer-scale diameters. Due to its three-dimensional feature and its high surface area-to-volume ratio, the fibrous structure 14 provides a high level of surface area to which cells may attach, thereby creating a stable network. In particular, the network formed by the fibrous structure 14 is less likely than naturally-formed fibrin to be broken down by enzymes present in the blood, and may occupy an aneurysm until host cells populate and synthesize a new natural matrix to fill the aneurysm.

The fibrous structure 14 is preferably coupled to the core member 12 by frictional contact between the fibrous structure 14 and the outer surface of the core member 12. The surface of the core member 12 may be textured to improve coupling between the fibrous structure 14 and the core member 12. The core member 12 may also include one or more transverse openings along the length of the core member 12, through which strands of the fibrous structure 14 can wrap to secure the fibrous structure 14 to the core member 12. Alternatively, the core member 12 may also include protrusions along the length of the core member 12, around which strands of the fibrous structure 14 can wrap or hook to secure the fibrous structure 14 to the core member 12. Alternatively, an adhesive, such as ultraviolet-curable adhesives, silicones, cyanoacrylates, and epoxies, may be used to secure the fibrous structure 14 to the core member 12. Furthermore, the fibrous structure 14 may be coupled to the core member 12 by chemical bonding between reactive groups on the fibrous structure 14 and the core member 12; fusing both materials so that they melt together; or temporarily melting the surface of the core member 12 to embed strands of the fibrous structure 14.

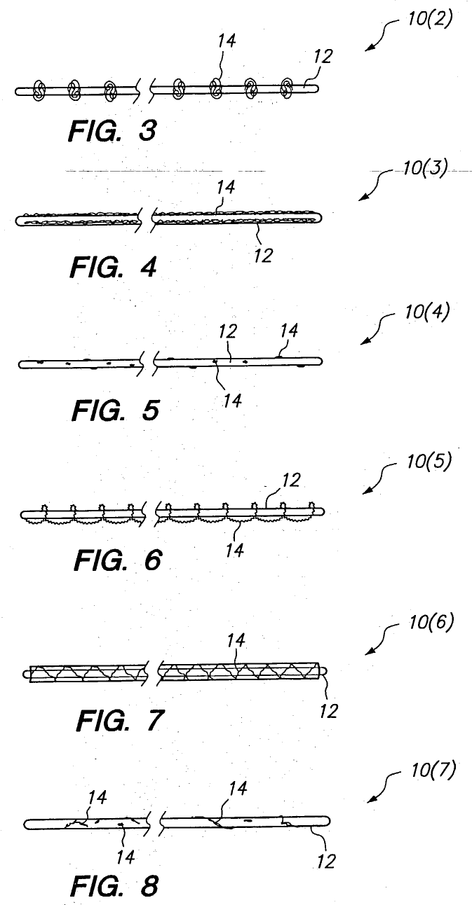

FIG. 1 shows an embodiment of the device 10(1) that includes a fibrous structure 14 carried by the core member 12. The fibrous structure 14 may be secured to the core member 12 by any of the methods discussed previously. As shown in FIG. 1, the fibrous structure 14 covers the core member 12 substantially along its entire length. However, such needs not to be the case, and the scope of this invention should not be so limited. For example, FIG. 3 is a side view of a vaso-occlusive device 10(2) that includes a plurality of sets of the fibrous structure 14 spaced intermittently along the length of the core member 12. The fibrous structure 14 may or may not be disposed completely around the circumference or periphery of the core member 12 at a point along the length of the core member 12, and it is a matter of design choice. FIG. 4 shows a vaso-occlusive device 10(3) that includes one or more fibrous structure 14 disposed axially along the length, and partially around the circumference, of the core member 12. As shown in FIG. 5, the fibrous structure 14 may also form one or more isolated patches with a defined shape and size that may be uniformly or randomly disposed on the surface of the core member 12. FIG. 6 shows another vaso-occlusive device 10(5), in which the fibrous structure 14 forms one or more spirals that extend helically around the core member 12. FIG. 7 shows yet another vaso-occlusive device 10(6), for which the fibrous structure 14 forms a mesh having a uniform grid pattern that is disposed around the core member 12. FIG. 8 shows a vaso-occlusive device 10(7), for which one or more fibrous structures 14 having random shapes are disposed randomly on the core member 12. It should be noted that other patterns or configurations for the fibrous structure 14 may be provided on the surface or around the core member 12.

The vaso-occlusive device 10 shown in the above-described embodiments generally has a substantially rectilinear (straight) or a curvilinear (slightly curved, i.e. having less than 360° spiral) relaxed configurations. Such vaso-occlusive devices may assume folded configurations when they are subjected to an external force (e.g., compressive forces generated when they are pushed against an object, such as the wall of an aneurysm). The vaso-occlusive device may also assume a variety of secondary and tertiary shapes or relaxed configurations, as will be discussed in further details below. For a vaso-occlusive device that has a secondary or a tertiary shape, the core member 12 is preferably made from a material that is more resilient, so as to provide rigidity to the vaso-occlusive device. The space-filling capacity of these vaso-occlusive devices is inherent within the secondary or tertiary relaxed shapes of these devices. When vaso-occlusive devices having secondary and/or tertiary shapes incorporate the fibrous structure 14 described herein, the devices provide a stable scaffold that can occlude an aneurysm, as discussed previously.

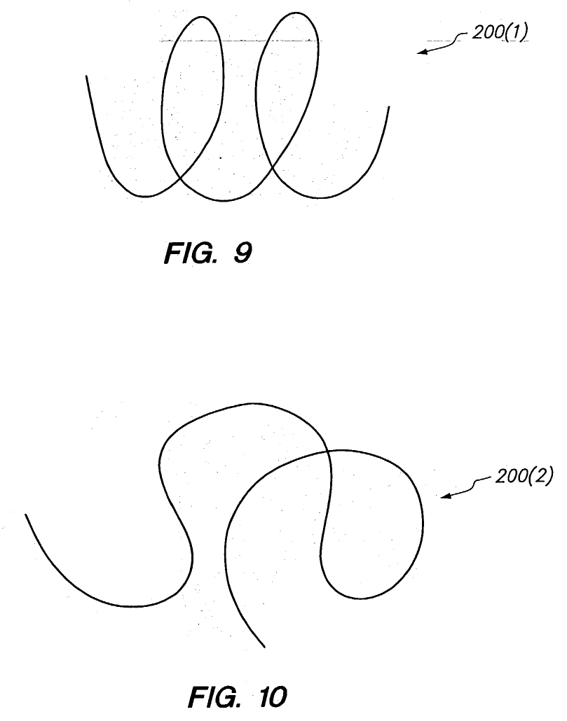

FIGS. 9 and 10 illustrate vaso-occlusive devices 200 having secondary shapes. These shapes are simply indicative of the various secondary shapes that may be used, and other shapes may be used as well. The device 200 illustrated in each of the FIGS. 9 and 10 includes the fibrous structure 14 as described previously, but is not shown for clarity.

FIG. 9 depicts a vaso-occlusive device 200(1) having a secondary shape of a helical coil. The helical coil may have an open pitch, such as that shown in FIG. 9, or a closed pitch. FIG. 10 illustrates a vaso-occlusive device 200(2) having a random secondary shape. Each of the secondary shapes shown in FIGS. 9 and 10 may be achieved by wrapping a core member 12 having a primary shape that is substantially linear, such as that shown in FIG. 1, around a mandrel, stylet, or other shaping element. The device 200 may optionally be heat treated, as known to one skilled in the art, to set the device into a secondary shape. It should be noted that the formation of vaso-occlusive devices into secondary shapes is well known in the art, and need not be described in further detail.

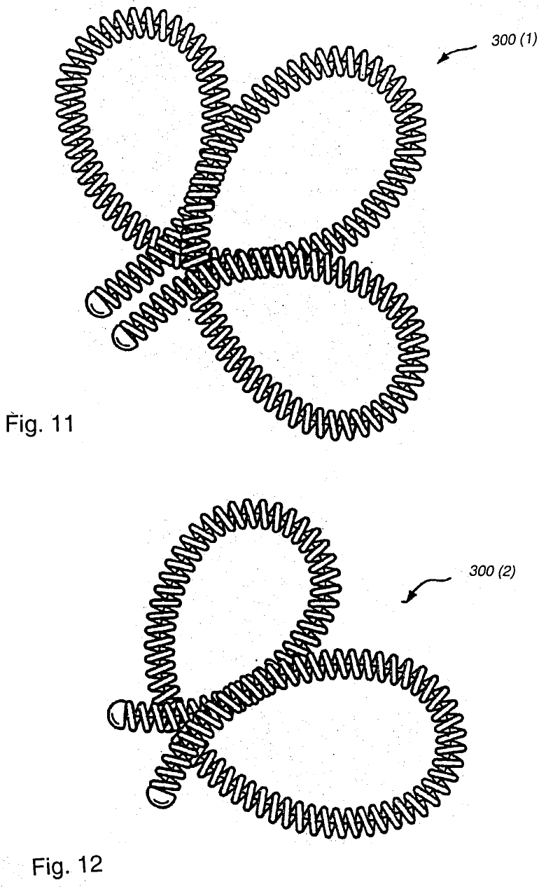

FIGS. 11-17 illustrate various vaso-occlusive devices 300 of this invention having a secondary shape of a helical coil, such as that shown in FIG. 9, and a tertiary shape. These shapes are simply indicative of the various tertiary shapes that may be used, and other shapes may be used as well. While not shown, the devices 300 illustrated in each of the FIGS. 11-17 include the fibrous structure 14, as discussed previously.

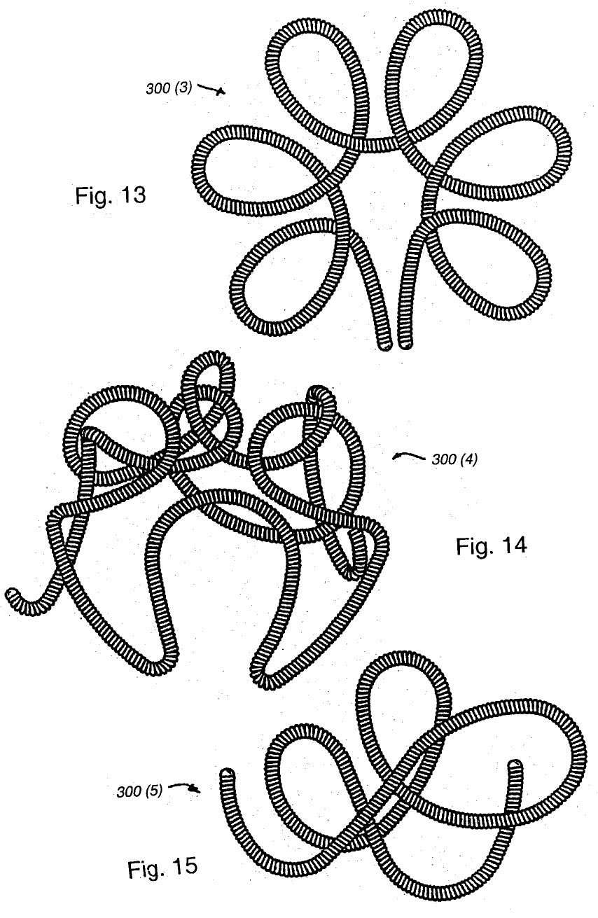

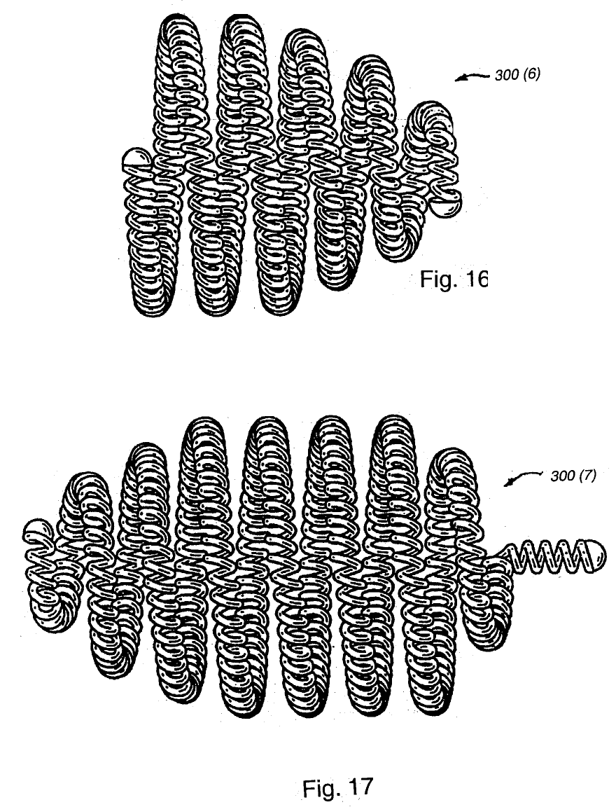

FIG. 11 depicts a device 300(1) having a tertiary shape of a clover leaf. FIG. 12 depicts a device 300(2) having a tertiary shape of a twisted figure-8. FIG. 13 depicts a device 300(3) having a flower-shaped tertiary shape. FIG. 14 depicts a device 300(4) having a substantially spherical tertiary shape. FIG. 15 illustrates a device 300(5) having a random tertiary shape. FIG. 16 illustrates a device 300(6) having a tertiary shape of a vortex. FIG. 17 illustrates a device 300(7) having a tertiary shape of an ovoid. It should be noted that vaso-occlusive device 10 may also have other secondary and tertiary shapes, and that it should not be limited to the examples illustrated previously. For example, the core member 12, and accordingly, the vaso-occlusive device, may be selectively sized to fill a particular aneurysm.

To make a tertiary shaped vaso-occlusive device 300, a core member 12 having a primary shape that is substantially rectilinear or curvilinear may be wrapped around a mandrel or other shaping element to form a secondary shape, such as the helical coil shown in FIG. 9. The core member 12 may be heat treated to shape the core member 12 into the secondary shape, as discussed previously. The secondary shaped vaso-occlusive member, such as the helical coil devices shown in FIG. 9, may then be wrapped around another shaping element to produce the tertiary shape. The core member 12 may be heat treated to form the tertiary shape. Stable coil designs, and methods of making them, are described in U.S. Pat. No. 6,322,576B1 to Wallace et al., the disclosure of which is expressly incorporated herein by reference. It should be noted that forming vaso-occlusive devices into tertiary shapes is well known in the art, and need not be described in further detail.

Although the previously described embodiments show that the core member 12 has an elongate shape, the scope of the invention should not be so limited. The core member 12 may also have other shapes, such as spherical, elliptical, or other design shapes. The core member 12 may also be an expandable member, such as a wire basket or an inflatable balloon, that is adapted to be placed within a body cavity.

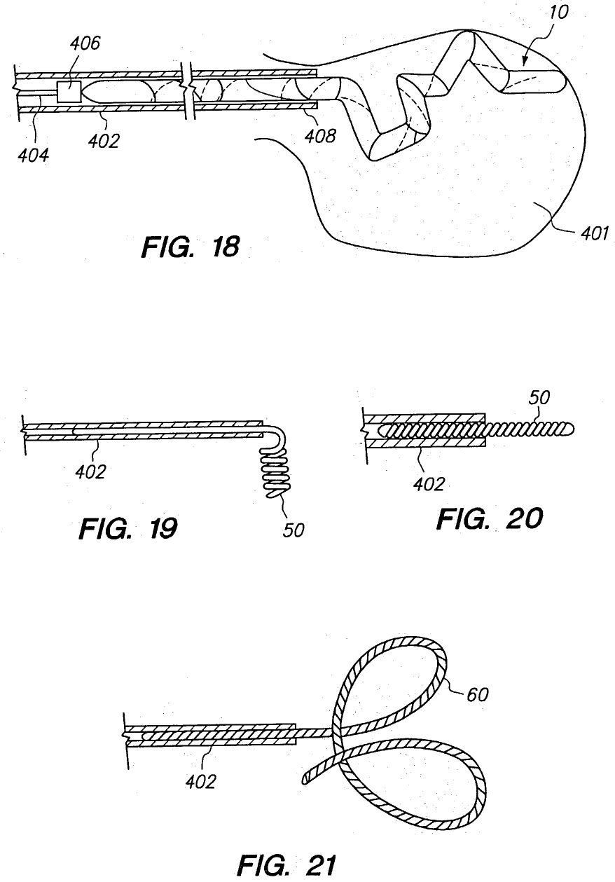

The method of using the previously described vaso-occlusive devices will now be discussed with reference to FIGS. 18-21. First, a delivery catheter 402 is inserted into the body of a patient. Typically, this would be through a femoral artery in the groin. Other entry sites sometimes chosen are found in the neck, for example, and are in general well known by physicians who practice these types of medical procedures. The delivery catheter 402, which may be a microcatheter or a sheath, may be positioned so that the distal tip 408 of the delivery catheter 402 is appropriately situated, e.g., within the mouth of the body cavity 401 to be treated. The insertion of the delivery catheter 402 may be facilitated by the use of a guidewire and/or a guiding catheter, as is known in the art. In addition, the movement of the catheter 402 may be monitored, for example, using fluoroscopy, ultrasound, and the like.

Once the delivery catheter 402 is in place, the vaso-occlusive device 10 is then inserted from the proximal end (not shown) of the delivery device 402, and into the lumen of the delivery device 402. This step is not necessary if the vaso-occlusive device 10 is already pre-loaded into the delivery catheter 402. For a vaso-occlusive device 10, such as those shown in FIGS. 1 and 3-8, that has no secondary or tertiary relaxed shape, the vaso-occlusive device 10 would naturally assume a substantially rectilinear or a curvilinear configuration when disposed within the lumen of the delivery device 402, without being subjected to a substantial stress.

For vaso-occlusive devices having secondary shape and/or tertiary shapes, such as the vaso-occlusive devices shown in FIGS. 9-17, they may be “stretched” to a substantially linear shape while residing within the lumen of the delivery catheter 402, as illustrated with the vaso-occlusive device 50 in FIG. 19. The advantage of having the vaso-occlusive devices assume a linear shape within the delivery device 402 is that the cross-sectional dimension of the delivery catheter 402 may be minimized, which may facilitate advancing the catheter 402 through tortuous or narrow arteries of a patient.

Alternatively, as shown in FIG. 20, a vaso-occlusive device having a secondary shape of a helical coil, such as the vaso-occlusive device 200 of FIG. 9, may be disposed within the lumen of a delivery catheter 402 in its unstretched configuration, as discussed previously with reference to FIG. 20. Furthermore, as shown in FIG. 21, a vaso-occlusive device having a tertiary shape made of a helical coil, such as any of the vaso-occlusive devices 300 shown in FIGS. 11-17, may be “stretched” to its secondary shape, in the form of a substantially linear helical coil, when disposed within the lumen of a delivery catheter 402.

Referring back to FIG. 18, the vaso-occlusive device 10 is preferably advanced distally towards the distal end 408 of the delivery catheter 402 using a core wire or pusher member 404. A plunger 406 may be attached to the distal end of the wire 404 to advance the vaso-occlusive device 10. Alternatively, fluid pressure may also be used to advance the vaso-occlusive device 10 along the delivery catheter 402. The inner diameter of the delivery catheter 402 should be made large enough to advance the vaso-occlusive device 10. On the other hand, the inner diameter of the delivery catheter 402 should not be significantly larger than the overall cross-sectional dimension of the vaso-occlusive device 10 in order to avoid bending and/or kinking the vaso-occlusive device 10 within the lumen of the delivery catheter 402.

For a vaso-occlusive device having no secondary or tertiary relaxed shape, the vaso-occlusive device may remain substantially rectilinear or curvilinear without undergoing substantial stress while residing within the lumen of the delivery catheter 402. Once the vaso-occlusive device 10 or a portion of the vaso-occlusive device 10 exits from the distal end 408 of the delivery catheter 402, it may remain substantially rectilinear or curvilinear until it contacts an object, e.g., the wall of the body cavity 401. If the vaso-occlusive device 10 is advanced further into the body cavity, the vaso-occlusive device 10 may buckle due to the continued advancing force. As a result, the vaso-occlusive device 10 may fold to assume a three-dimensional structure within the aneurysm. For vaso-occlusive devices having secondary or tertiary shapes, the vaso-occlusive device may be biased to resume its relaxed configuration when ejected from the lumen of the delivery catheter 402. The shape of the secondary or tertiary relaxed configuration may help fill up the body cavity 401.

Additional vaso-occlusive devices 10 may also be placed within the body cavity 401 by repeating the relevant steps discussed above. When a desired number of vaso-occlusive devices has been placed within the body cavity 401, the delivery catheter 402 may be withdrawn from the body cavity 401 and the patient's body. Once the vaso-occlusive devices are deployed in the body cavity 401, an embolism is formed therein to occlude the body cavity 401.

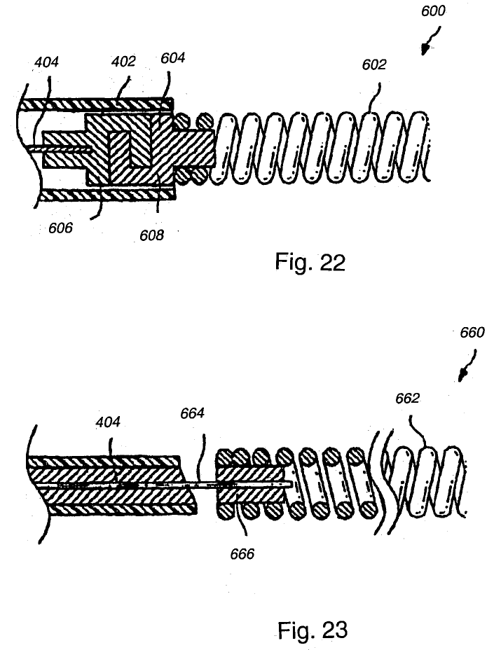

FIG. 22 depicts an embodiment, generally designated 600, having a vaso-occlusive device 602 that may be deployed from a catheter, such as the delivery catheter 402 discussed previously, through operation of a connective joint 604. The vaso-occlusive device 602 may be any of the devices depicted in FIGS. 1 and 3-17, i.e., including the fibrous structure 14 (not shown for clarity). Joint 604 has a clasp section 606 that may remain attached to the core wire 404 when the sheath or catheter body 402 is retracted proximally. Joint 604 also may occlusive device 602 and interlocking with clasp section 606 when the assembly is within the sheath 402. When the sheath 402 is withdrawn from about the assembly, the clasp sections may disengage, thereby detaching the vaso-occlusive device 602.

The vaso-occlusive devices described herein may also be detachable by an electrolytic joint or connection such as described in U.S. Pat. Nos. 5,234,437, 5,250,071, 5,261,916, 5,304,195, 5,312,415, and 5,350,397, the disclosure of which is expressly incorporated by reference herein.

FIG. 23 shows an embodiment, generally designated 660, having a vaso-occlusive device 662 that may be detached using a connective joint 664 that is susceptible to electrolysis. The vaso-occlusive device 662 may be any one of the devices depicted in FIGS. 1 and 3-17, and may include the fibrous structure 14 (not shown for clarity). Such joints are described in detail in U.S. Pat. Nos. 5,423,829, 6,165,178, and 5,984,929, the disclosures of which are expressly incorporated by reference herein. Joint 664 may be made of a metal which, upon application of a suitable voltage to a core wire 404, may erode in the bloodstream, thereby releasing the vaso-occlusive device 662. The vaso-occlusive device 662 may be made of a metal that is more “noble” in the electromotive series than the joint 664. A return electrode (not shown) may be supplied to complete the circuit. The region of core wire 404 proximal to the joint is insulated to focus the erosion at the joint. A bushing 666 may be used to connect the distal end of core wire 404 to the proximal end of the vaso-occlusive device 662. To deploy the vaso-occlusive device 662, the vaso-occlusive device 662 attached to the core wire 404 is first placed within a body cavity. An electric current is then applied to the core wire 404 to dissolve the connective joint 664, thereby detaching the vaso-occlusive device 662 from the core wire 404. It should be noted that methods of delivering vaso-occlusive devices by electrolytic disintegration of a core wire joint are well known in the art, and need not be described in further detail.

Thus, although several preferred embodiments have been shown and described, it would be apparent to those skilled in the art that many changes and modifications may be made thereunto without the departing from the scope of the invention, which is defined by the following claims and their equivalents.

What is claimed:

1. A vaso-occlusive device, comprising:
a core member; and
a fibrous structure carried by the core member, the fibrous structure comprises one or more strands of nanofibers.

2. The vaso-occlusive device of claim 1, wherein the fibrous structure is a product generated at least in part by an electrospinning process comprises the steps of:
supplying a polymer solution through a needle;
electrostatically charging the needle;
electrostatically charging a metal plate that is placed at a distance from the needle, the metal plate having a charge that is opposite that of the needle, thereby sending a jet of the polymer solution towards the metal plate; and
collecting the fibrous structure from the metal plate.

3. The vaso-occlusive device of claim 2, wherein the polymer solution comprises a material selected from a group consisting of polyethylene oxide, acrylic, nylon, polyethylene glycol, polyacrylonitrile, polyethylene terephthalate, PPTA, polyglycolic acid, polylactic acid, protein, polysaccharide, PLGA, polycaprolactone, polyhydroxybutyrate, polyhydroxyvalerate, polydioxanone, polycarbonates, polyanhydrides, polyhydroxyalkanoates, polyarylates, and polyamino acids.

4. The vaso-occlusive device of claim 2, wherein the polymer solution is prepared by a process comprising the steps of:
dissolving 1 g of PLGA in 20 mL of organic solvent mixture, the mixture comprises tetrahydrofuran and dimethylformamide; and
vortexing the mixture overnight.

5. The vaso-occlusive device of claim 1, wherein the fibrous structure is made from a material selected from a group consisting of polyethylene oxide, acrylic, nylon, polyethylene glycol, polyacrylonitrile, polyethylene terephthalate, PPTA, polyglycolic acid, polylactic acid, protein, polysaccharide, PLGA, polycaprolactone, polyhydroxybutyrate, polyhydroxyvalerate, polydioxanone, polycarbonates, polyanhydrides, polyhydroxyalkanoates, polyarylates, polyamino acids, and co-polymers thereof.

6. The vaso-occlusive device of claim 1, wherein the fibrous structure comprises a bioactive agent.

7. The vaso-occlusive device of claim 6, wherein the bioactive agent is selected from the group consisting of cytokines, extracellular matrix molecules, matrix metalloproteinase inhibitors, trace metals, molecules that stabilize thrombus formation or inhibit clot lysis, P1 epitope of fibrin, P2 epitope of fibrin, nucleic acids, and functional fragments thereof.

8. The vaso-occlusive device of claim 1, wherein the nanofibers have diameters or cross-sectional dimensions between about 100 nm and 5000 nm.

9. The vaso-occlusive device of claim 1, wherein the fibrous structure has an architecture that is similar to that of a natural extracellular matrix.

10. The vaso-occlusive device of claim 1, wherein the fibrous structure is disposed completely around a periphery of the core member.

11. The vaso-occlusive device of claim 10, wherein the core member has an overall cross-sectional dimension between about 0.01 inch and 0.015 inch.

12. The vaso-occlusive device of claim 1, wherein the fibrous structure is disposed at least partially around a circumference of the core member.

13. The vaso-occlusive device of claim 1, wherein the one or more strands of fibers are spaced intermittently along a length of the core member.

14. The vaso-occlusive device of claim 1, wherein the one or more strands of nanofibers form a mesh defining a grid pattern around the core member.

15. The vaso-occlusive device of claim 1, wherein the core member has a substantially rectilinear relaxed configuration.

16. The vaso-occlusive device of claim 1, wherein the core member has a curvilinear relaxed configuration.

17. The vaso-occlusive device of claim 1, wherein the core member has a secondary relaxed configuration.

18. The vaso-occlusive device of claim 17, wherein the core member is a helical coil.

19. The vaso-occlusive device of claim 1, wherein the core member has a tertiary relaxed configuration.

20. The vaso-occlusive device of claim 19, wherein the core member has a twisted-8 shape.

21. The vaso-occlusive device of claim 19, wherein the core member has a spherical shape.

22. The vaso-occlusive device of claim 1, wherein the core member has an end that is detachably coupled to a core wire.

23. The vaso-occlusive device of claim 22, wherein the core member is detachably coupled to the core wire by an electrolytic joint.

24. The vaso-occlusive device of claim 22, wherein the core member is detachably coupled to the core wire by a mechanical joint.

25. The vaso-occlusive device of claim 1, wherein the core member comprises an expandable member.

26. The vaso-occlusive device of claim 25, wherein the expandable member is a balloon.

27. The vaso-occlusive device of claim 1, wherein the fibrous structure is coupled to the core member by surface friction.

28. The vaso-occlusive device of claim 1, wherein a surface of the core member is textured.

29. The vaso-occlusive device of claim 1, wherein the core member includes one or more protrusions around which one or more strands of the nanofibers can wrap or hook to secure the fibrous structure to the core member.

30. The vaso-occlusive device of claim 1, wherein the fibrous structure is secured to the core member by an adhesive selected from the group consisting of ultraviolet-curable adhesive, silicone, cyanoacrylate, and epoxy.

31. The vaso-occlusive device of claim 1, wherein the fibrous structure is secured to the core member by a chemical bonding between reactive groups on the fibrous structure and the core member.

32. The vaso-occlusive device of claim 1, wherein one or more of the nanofibers are at least partially embedded below a surface of the core member.

33. The vaso-occlusive device of claim 1, wherein the fibrous structure and the core member are fused together.

34. The vaso-occlusive device of claim 1, wherein the core member comprises a bioactive agent.

35. The vaso-occlusive device of claim 34, wherein the bioactive agent is selected from the group consisting of cytokines, extracellular matrix molecules, matrix metalloproteinase inhibitors, trace metals, molecules that stabilize thrombus formation or inhibit clot lysis, P1 epitope of fibrin, P2 epitope of fibrin, nucleic acids, and functional fragments thereof.

36. A catheter assembly for occluding a body cavity, comprising:
a delivery catheter having a distal end, a proximal end, and a lumen extending there between; and
a vaso-occlusive device deliverable through the lumen of the delivery catheter, the vaso-occlusive device having a core member and a fibrous structure carried by the core member, the fibrous structure comprises one or more strands of nanofibers.

37. The catheter assembly of claim 36, wherein the fibrous structure is a product generated at least in part by an electrospinning process comprises the steps of:
supplying a polymer solution through a needle;
electrostatically charging the needle;
electrostatically charging a metal plate that is placed at a distance from the needle, the metal plate being in a charge that is opposite that of the needle, thereby sending a jet of the polymer solution towards the metal plate; and
collecting the fibrous structure from the metal plate.

38. The catheter assembly of claim 37, wherein the polymer solution comprises a material selected from a group consisting of polyethylene oxide, acrylic, nylon, polyethylene glycol, polyacrylonitrile, polyethylene terephthalate, PPTA, polyglycolic acid, polylactic acid, protein, polysaccharide, PLGA, polycaprolactone, polyhydroxybutyrate, polyhydroxyvalerate, polydioxanone, polycarbonates, polyanhydrides, polyhydroxyalkanoates, polyarylates, polyamino acids, and co-polymers thereof.

39. The catheter assembly of claim 37, wherein the polymer solution is prepared by a process comprising the steps of:
dissolving 1 g of PLGA in 20 mL of organic solvent mixture, the mixture comprises tetrahydrofuran and dimethylformamide; and
vortexing the mixture overnight.

40. The catheter assembly of claim 36, wherein the fibrous structure is made from a material selected from a group consisting of polyethylene oxide, acrylic, nylon, polyethylene glycol, polyacrylonitrile, polyethylene terephthalate, PPTA, polyglycolic acid, polylactic acid, protein, polysaccharide, PLGA, polycaprolactone, polyhydroxybutyrate, polyhydroxyvalerate, polydioxanone, polycarbonates, polyanhydrides, polyhydroxyalkanoates, polyarylates, polyamino acids, and co-polymers thereof.

41. The catheter assembly of claim 40, wherein the fibrous structure is made from PLGA.

42. The catheter assembly of claim 36, wherein the fibrous structure comprises a bioactive agent.

43. The catheter assembly of claim 42, wherein the bioactive agent is selected from the group consisting of cytokines, extracellular matrix molecules, matrix metalloproteinase inhibitors, trace metals, molecules that stabilize thrombus formation or inhibit clot lysis, P1 epitope of fibrin, P2 epitope of fibrin, nucleic acids, and functional fragments thereof.

44. The catheter assembly of claim 36, wherein the nanofibers have diameters or cross-sectional dimensions that range from 100 to 5000 nm.

45. The catheter assembly of claim 36, wherein the fibrous structure has an architecture that is similar to that of a natural extracellular matrix.

46. The catheter assembly of claim 36, wherein the fibrous structure is disposed completely around a circumference of the core member.

47. The catheter assembly of claim 46, wherein the fibrous structure has an overall diameter that ranges from 0.01 inch to 0.015 inch.

48. The catheter assembly of claim 36, wherein the fibrous structure is disposed partially around a circumference of the core member.

49. The catheter assembly of claim 36, wherein the fibrous structure is disposed intermittently along a length of the core member.

50. The catheter assembly of claim 36, wherein the fibrous structure forms a mesh having a uniform grid pattern that is disposed around the core member.

51. The catheter assembly of claim 36, wherein the core member has a substantially linear relaxed configuration.

52. The catheter assembly of claim 36, wherein the core member has a curvilinear relaxed configuration.

53. The catheter assembly of claim 36, wherein the core member has a secondary relaxed configuration.

54. The catheter assembly of claim 53, wherein the core member is a helical coil.

55. The catheter assembly of claim 36, wherein the core member has a tertiary relaxed configuration.

56. The catheter assembly of claim 55, wherein the core member has a twisted-8 shape.

57. The catheter assembly of claim 55, wherein the core member has a spherical shape.

58. The catheter assembly of claim 55, wherein the core member is sized to fit within an aneurysm.

59. The catheter assembly of claim 36, wherein the core member has an end that is detachably coupled to a core wire.

60. The catheter assembly of claim 59, wherein the core member is detachably coupled to the core wire by an electrolytic joint.

61. The catheter assembly of claim 59, wherein the core member is detachably coupled to the core wire by a mechanical joint.

62. The catheter assembly of claim 36, wherein the fibrous structure is coupled to the core member by surface friction.

63. The catheter assembly of claim 36, wherein a surface of the core member is textured.

64. The catheter assembly of claim 36, wherein the core member includes one or more protrusions around which one or more strands of nanofibers can wrap or hook to secure the fibrous structure to the core member.

65. The catheter assembly of claim 36, wherein the fibrous structure is secured to the core member by an adhesive selected from the group consisting of ultraviolet-curable adhesive, silicone, cyanoacrylate, and epoxy.

66. The catheter assembly of claim 36, wherein the fibrous structure is secured to the core member by a chemical bonding between reactive groups on the fibrous structure and the core member.

67. The catheter assembly of claim 36, wherein one or more of the nanofibers are at least partially embedded below a surface of the core member.

68. The catheter assembly of claim 36, wherein the fibrous structure and the core member are fused together.

69. The catheter assembly of claim 36, wherein the core member comprises a bioactive agent.

70. The catheter assembly of claim 69, wherein the bioactive agent is selected from the group consisting of cytokines, extracellular matrix molecules, matrix metalloproteinase inhibitors, trace metals, molecules that stabilize thrombus formation or inhibit clot lysis, P1 epitope of fibrin, P2 epitope of fibrin, nucleic acids, and functional fragments thereof.

71. A method of manufacturing a vaso-occlusive device, comprising:
supplying a polymer solution through a needle;
electrostatically charging the needle;
electrostatically charging a metal plate that is placed at a distance from the needle, the metal plate being in a charge that is opposite that of the needle, thereby sending a jet of the polymer solution towards the metal plate;
collecting fibers from the metal plate; and
coupling the one or more of the fibers to a surface of a core member.

72. The method as in claim 71, wherein the polymer solution comprises a material selected from a group consisting of polyethylene oxide, acrylic, nylon, polyethylene glycol, polyacrylonitrile, polyethylene terephthalate, PPTA, polyglycolic acid, polylactic acid, protein, polysaccharide, PLGA, polycaprolactone, polyhydroxybutyrate, polyhydroxyvalerate, polydioxanone, polycarbonates, polyanhydrides, polyhydroxyalkanoates, polyarylates, polyamino acids, and co-polymers thereof.

73. The method as in claim 71, further comprising preparing the polymer solution.

74. The method as in claim 73, wherein the preparing comprises the steps of:
dissolving 1 g of PLGA in 20 mL of organic solvent mixture, the mixture comprises tetrahydrofuran and dimethylformamide; and
vortexing the mixture overnight.

75. The method as in claim 71, wherein the electrostatically charging the needle and the electrostatically charging the metal plate comprise using a voltage generator.

76. The method as in claim 71, wherein the metal plate comprises copper.

77. The method as in claim 71, wherein the coupling comprises securing the one or more of the fibers on a surface of the core member by an adhesive.

78. The method as in claim 71, wherein the coupling comprises maintaining a frictional contact at an interface between the one or more of the fibers and a surface of the core member.

79. The method as in claim 71, wherein the coupling comprises wrapping one or more of the fibers around a protrusion of the core member.

80. The method as in claim 71, wherein the coupling comprises chemical bonding between reactive groups on the fibers and the core member.

81. The method as in claim 71, wherein the coupling comprises embedding at least a part of one or more of the fibers below a surface of the core member.

82. The method as in claim 71, wherein the coupling comprises fusing one or more of the fibers and the core member together.

83. A method of occluding a body cavity, comprising:
providing a delivery catheter carrying a vaso-occlusive device, the vaso-occlusive device having a core member and a fibrous structure coupled to the core member, the fibrous structure comprises strands of nanofibers;
positioning the delivery catheter adjacent to an opening of the body cavity;
advancing the vaso-occlusive device within the lumen of the delivery catheter until the vaso-occlusive device exits from the delivery catheter.

84. The method as in claim 83, wherein the advancing comprises using a core wire.

85. The method as in claim 83, wherein the advancing comprises using fluid pressure.

86. The method of claim 83, wherein the body cavity comprises an aneurysm.
